(19) United States
(12) Patent Application Publication
(10) Pub. No.: US 2016/0038333 A1
(43) Pub. Date: Feb. 11, 2016

(54) Sleep Aid Apparatus

(71) Applicant: Rebecca Tyke, Grand Rapids, MI (US)

(72) Inventor: Rebecca Tyke, Grand Rapids, MI (US)

(21) Appl. No.: 14/818,558

(22) Filed: Aug. 5, 2015

Related U.S. Application Data
(60) Provisional application No. 62/033,298, filed on Aug. 5, 2014.

(51) Int. Cl. A61F 5/37

(52) U.S. Cl. CPC A61F 5/37

(57) ABSTRACT

A sleep aid apparatus that includes a harness member and a movement limiting node. The harness member includes a body having a first end and a second end, and a closure that couples the two ends together to form a hoop that can be positioned about the body of a user around the region of the waist. The movement limiting node is positionable along the harness member between the first and second ends. A plurality of movement limiting nodes can be provided that are each independently moved along the harness member.

### CROSS-REFERENCE TO RELATED APPLICATION

The present application claims priority from U.S. Provisional Patent App. Ser. No. 62/033,298, entitled “Sleep Aid Apparatus”, filed Aug. 5, 2014, the entire specification of which is hereby incorporated by reference in its entirety.

### BACKGROUND OF THE DISCLOSURE

1. Field of the Disclosure

The invention relates in general to health aids and apparatuses, and more particularly, to a sleep aid apparatus which is configured to be positioned about the waist region of a user to assist with the user being in a desired position when sleeping.

2. Background Art

It is known that for certain individuals it is desirable to have the user sleeping in a particular orientation. That is, the user may need to rest on a particular side of the body, or the front or back of the body, when sleeping due to injury, a particular condition or the like. In many instances, it is difficult for the user to remain in the desired position throughout the period of sleep.

Certain solutions, such as pillows, or other obstructions have been utilized which attempt to urge and/or maintain the user in the desired orientation. These solutions have met limited success. That is, it is often the case that a sleeping, or half asleep, individual will dislodge or move the obstruction out of the way in order to reorientate. As such, the utility is limited to those situations wherein the user maintains the obstructions in the proper orientation.

Other solutions have placed an emphasis on rigid bodies that can be coupled to a user. Problematically, such devices lack the necessary adjustment that can facilitate comfort to a particular user. Additionally, these devices cannot be adjusted so that a user can select a different orientation.

### SUMMARY OF THE DISCLOSURE

The disclosure is directed to a harness member and one or more movement limiting nodes. The harness member can be extended about the waist, abdomen and/or mid-section of a user such that the nodes preclude undesirable rolling over or movement.

The harness member comprises a body having a first end and a second end, as well as a closure that can couple the first and second ends together. The movement limiting nodes include an outward node surface, an inward node surface and a harness opening. The harness member is extended through the harness opening.

In the proper position, the movement limiting nodes are slidably positionable along the harness member between the first and second ends. When on the user, the inward node surface is sandwiched between the user and the harness member. At the same time, the outward node surface extends outwardly from the harness member opposite the inward node surface.

In an aspect of the disclosure, the disclosure is directed to a sleep aid apparatus comprising a harness member and at least one movement limiting member. The harness member includes a body with a first end and a second end and defines a length and a width. A closure member is configured to couple the first end and the second end in releasable engagement. The movement limiting node is positionable along the harness member, and includes an outward node surface extending from an outer surface of the body of harness member.

In some configurations, the at least one movement limiting node includes an outward node surface extending from an outer surface of the body of the harness member, and inward node surface extending from an inner surface of the body of the harness member.

In some configurations, a passageway is defined between the outward node surface and the inward node surface. The passageway is configured to receive the harness member therethrough.

In some configurations, the at least one movement limiting node releasably lockingly engages the harness member, to, in turn, preclude the relative movement of the at least one movement limiting node and the harness member.

In some configurations, the at least one movement limiting node comprises three movement limiting nodes that are positionable in a space apart orientation.

In some configurations, the outward node surface comprises a generally hemispherical configuration.

In some configurations, the closure member includes a first component positionable proximate the first end of the body of the harness member, and a second component positionable proximate the second end of the body of the harness member. The first component and the second component are releasably attachable to each other.

In some configurations, the at least one movement limiting node further comprises an outward node component, an inward node component and a latching mechanism. The outward node component has the outward node surface. The inward node component is pivotably coupled to the outward node component. The outward node component and the inward node component define a passageway therebetween for the harness member. Pivoting of the outward node component relative to the inward node component provides selective access to the passageway therebetween, and directs the outward node component and the inward node component between an open orientation and a harness member retaining orientation. The latching mechanism configured to releasably maintain the outward node component and the inward node component in a harness member retaining orientation.

In some configurations, the outward node component and the inward node component are pivotable about an axis that is substantially parallel to the passageway for the harness member.

In some configurations, the axis is substantially maintained within the footprint of the inward node component and the outward node component.

In some configurations, interference between the inward node component and the outward node component on a side opposite the axis from the passageway precludes further pivoting thereof away from the harness member retaining orientation.

In some configurations, the latching mechanism includes a pivotable latch protrusion on one of the inner node component and the outer node component, and a capture flange on the other of the inner node component and the outer node component.

In some configurations, the outward node component further includes at least one grasping protrusion that extends into the passageway which is configured to extend into the harness member when in the harness member retaining orientation.

In some configurations, the at least one movement limiting node further comprises an outward node, an inward node and a locking component. The outward node component includes the outward node surface, and an outer rim. The inward node component includes a lower outer rim, and an upper outer rim, and includes an inner bore extending therethrough. The inner bore is threaded along at least a portion thereof. The outward node is coupled to the inward node. The inward node and the outward node cooperatively form a passageway for the harness member. The locking component has a body with an upper control member and a depending cylindrical portion. The depending cylindrical portion includes threads on the outer surface thereof, and a lower edge opposite the upper control member. The depending cylindrical portion is threadedly coupled to the threads of the inner bore of the inward node component. Continued threading will direct the lower edge into contact with the harness member, sandwiching the harness member between the lower edge and the outward node component.

In some configurations, the outward node component includes a grasping protrusion positioned on the outward node component so as to correspond to the lower edge of the depending cylindrical portion, to, in turn, provide a point of contact with the lower edge.

In some configurations, a vibration element is positioned within the movement limiting node. The vibration element is activated by force upon the outward node component.

In some configurations, a communication element is coupled to the vibration element. The communication element configured to communicate any activation of the vibration element through a wireless communication.

### BRIEF DESCRIPTION OF THE DRAWINGS

The disclosure will now be described with reference to the drawings wherein:

FIG. 1 of the drawings is a side elevational view of the sleep aid apparatus of the present disclosure;

FIG. 2 of the drawings is a side elevational view of the movement limiting node as coupled to the harness member;

FIG. 3 of the drawings is a cross-sectional view of the movement limiting node, showing, in particular, the harness opening;

FIG. 4 of the drawings is a side elevational view of the movement limiting node of the present disclosure, showing the harness opening;

FIG. 5 of the drawings is an exploded view of an embodiment of the sleep aid apparatus of the present disclosure, showing, in particular, an alternate movement limiting node;

FIG. 6 of the drawing is a top plan view of the embodiment shown in FIG. 5;

FIG. 7 of the drawings is a cross-sectional view of the embodiment shown in FIG. 5, taken generally about lines D-D of FIG. 6;

FIG. 8 of the drawings is a side elevational view of the embodiment shown in FIG. 5;

FIG. 9 of the drawings is a cross-sectional view of the embodiment shown in FIG. 5, taken generally about lines A-A of FIG. 8;

FIG. 10 of the drawings is a cross-sectional view of the embodiment shown in FIG. 5, taken generally about lines C-C of FIG. 6;

FIG. 11 of the drawings is a detail cross-sectional view of the embodiment shown in FIG. 5, taken generally about the area E of FIG. 6;

FIG. 12 of the drawings is a perspective view of an embodiment of the sleep aid apparatus of the present disclosure, showing, in particular, an alternate movement limiting node;

FIG. 13 of the drawings is a top plan view of the embodiment shown in FIG. 12;

FIG. 14 of the drawings is a cross-sectional view of the embodiment shown in FIG. 12, taken generally about lines D-D of FIG. 13;

FIG. 15 of the drawings is a cross-sectional view of the embodiment shown in FIG. 12, taken generally about lines C-C of FIG. 13;

FIG. 16 of the drawings is a detail cross-sectional view of the embodiment shown in FIG. 12, taken generally about the detail in FIG. 15;

FIG. 17 of the drawings is a side elevational view of the embodiment shown in FIG. 12, showing, in particular, the open configuration;

FIG. 18 of the drawings is a cross-sectional view of the embodiment shown in FIG. 12, taken generally about lines A-A of FIG. 17;

FIG. 19 of the drawings is a side elevational view of the embodiment shown in FIG. 12;

FIG. 20 of the drawings is a cross-sectional view of the embodiment shown in FIG. 12, taken generally about lines F-F of FIG. 19;

FIG. 21 of the drawings is a schematic representation of an exemplary sleep aid apparatus of the present disclosure;

FIGS. 22a and 22b are schematic representations of the sleep aid apparatus as installed on a torso of a male and female body; and

FIG. 23a through 23c are schematic representations of the sleep aid apparatus in use, with the user positioning and orienting the sleep aid apparatus in three different configurations, to promote sleep on the user's back, the user's side and the user's stomach/front, respectively.

### DETAILED DESCRIPTION OF THE DISCLOSURE

While this invention is susceptible of embodiment in many different forms, there is shown in the drawings and described herein in detail a specific embodiment with the understanding that the present disclosure is to be considered as an exemplification and is not intended to be limited to the embodiment illustrated

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

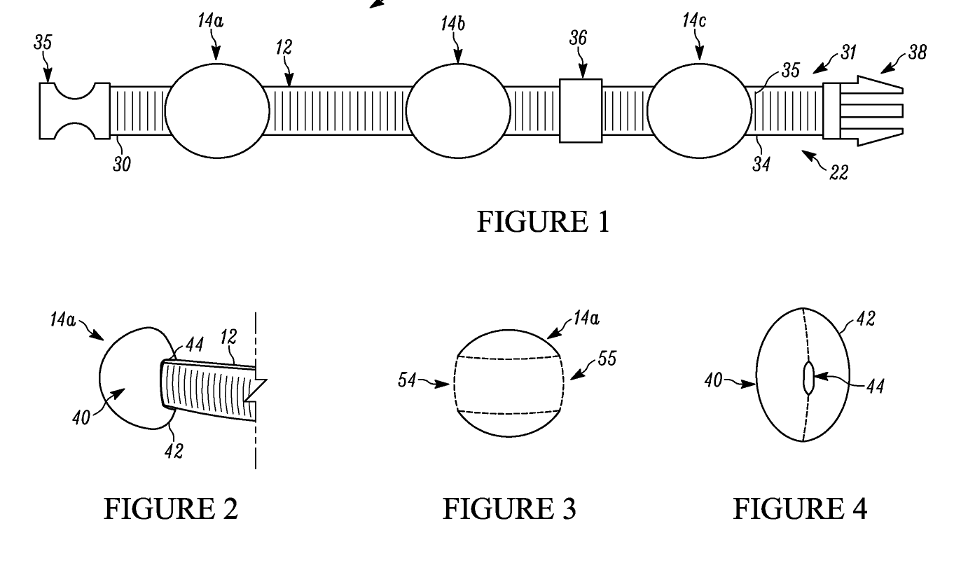

Referring now to the drawings and in particular to FIG. 1, the sleep aid apparatus is shown in FIG. 1 as comprising harness member 12, and movement limiting nodes 14a through 14c. As will be explained below, the sleep aid apparatus is configured to direct the user into a desired position, preclude a change in position, and/or to urge the user to maintain a desired position, all while sleeping. In addition, as will also be explained, the sleep aid apparatus is configured to be adjustable and so as to facilitate the positioning of the movement limiting nodes in different orientations, to allow for the use of additional nodes, and to allow for the use of differently shaped nodes.

More particularly, and referring now to the figures, the harness member 12 includes body 20 and closure 22. The body 20 includes first end 30, second end 31, first side 34 and second side 35. The first and second ends are separated from each other so as to define length 33 of the body. The first and second sides 34, 35 are separated from each other so as to define the width 32. An adjustment member 36 may be utilized, as is known in the art to alter the length of the body 20 and this member or a functional equivalent thereof may be incorporated into the closure 22. A number of different materials are contemplated, such as, for example, a woven nylon material. Of course, other materials, such as leather, synthetic polymers and woven fabrics or fibers are likewise contemplated for use. In the embodiment shown, the body is flexible and bendable (while typically being of a fixed length with little elasticity). Typically, the width of the body is substantially greater than the thickness thereof

The closure 22 is shown in FIG. 1 as comprising first component 38 and second component 39. The first component is positioned at the first end 30 of the body and the second component is positioned at the second end 31 of the body. The first component and the second component cooperate to releasably maintain the first end and the second end in operable secured engagement (although it will be understood that due to adjustment mechanisms, the second end or the first end, in this case shall mean the effective first and or second end after the shortening (i.e., the effective or operable ends of an adjusted body of the harness member). The user, can manipulate portions of the two components relative to each other to separate the two components. In other configurations, other types of members can be utilized. In particular, the in another embodiment, hook and loop fasteners may be utilized, wherein one of the components of the closure comprises the hook portion and the other of the components comprises the loop portion. In other embodiments, a belt and buckle system can be utilized. There are other fasteners (such as snaps and the like) which may also be utilized. Indeed, the disclosure is not limited to any particular configuration of the closure.

The movement limiting nodes 14a through 14c are selectively positionable along the length of the harness member, without limitation. It will be understood that the different movement limiting nodes 14a through 14c may be substantially identical in function, and for this reason, movement limiting node 14a will be described with the understanding that the movement limiting nodes 14b and 14c are substantially identical. It will also be understood that there may be variations in the movement limiting nodes, that additional nodes, or fewer nodes may be utilized to achieve the desired functions. In addition, it will be understood that the user may be able to selectively couple and decouple nodes from the harness member. It is contemplated that the nodes surround the harness member (with the harness member passing therebetween through a passageway. It is likewise contemplated that the nodes may have a connector or other item that extends through the harness member. In further embodiments, it is contemplated that only an outward node component or surface is provided.

An exemplary movement limiting node 14a is shown as having outward node surface 40, inward node surface 42 and harness opening 44. In general terms, the node, in the configuration shown comprises a generally spherical member with the harness opening extending through the spherical member so as to split the node into a portion that is on the outside of the node (the outward node surface) and a portion that is between the harness and the body of a user (inward node surface), when positioned in the operable configuration. It will be understood that the node is not limited to a spherical shape, and even where spherical, the two portions may or may not be the same in configuration. The use of a spherical node and the positioning of the harness opening is exemplary solely.

The outward node surface includes top 50, bottom 51, first side 52 and second side 53. As identified above, the outward node surface comprises a substantially hemispherical configuration that extends away from the harness opening and the body of the user when in the operable configuration. The outward node surface protrudes away from the harness opening sufficiently to preclude a user from rolling beyond the node. The first side and the second side are both defined by the boundaries with the harness opening 44.

The inward node surface in the configuration also comprises a hemispherical portion, although a flattened portion or a portion that is different than the outward node surface is likewise contemplated. The inward node surface is sandwiched between the harness member and the body of the user when in use. Therefore, the configuration of the inward node surface is geared toward enhancing the comfort of the user wearing the device.

The harness opening 44 extends through the movement limiting node 14a from one side to the other side of the outward node surface. In the configuration shown, the harness opening is generally positioned between the top and bottom ends of the outward node surface, although such a position is not required. The harness opening comprises an opening that extends from first end 54 to second end 55. The harness opening is generally configured to match the shape of the harness member sufficiently closely to allow the sliding of the movement limiting node along the harness member with the application of a force, but to resist inadvertent movement thereagainst. Thus, the node positions can be manually altered through imparting a force upon the node, while inadvertent and undesired movement is generally precluded.

As stated above, the remaining nodes 14b and 14c are substantially identical to the node 14a, in the configuration shown. As such, the nodes 14b and 14c have substantially the same structure. Of course, there may be variations between the different nodes.

It will be understood that the nodes may be slid off the harness member or threaded onto the harness member so as to alter the number of nodes that can be utilized. Additionally, in embodiments having relatively larger closure components at the ends of the harness member, the nodes may have a slit or channel that provides communication from the outside surface to the harness opening so that the harness member can be inserted through the slit or channel into the harness opening.

Additionally, the nodes may comprise a multiple part node which is coupled together to sandwich the harness therebetween. The two components may be rotated relative to each other to lock them in place. In other embodiments, the node can be releasably coupled to the belt through different configurations. In still other configurations, the nodes can be extended about the harness member and then through the use of fasteners, attached onto itself to form a loop that extends around the harness member. In still other configurations, it is contemplated that the sleep aid apparatus is comfortable when wearing in a desired sleep position, while the nodes provide discomfort when in an undesirable sleep position.

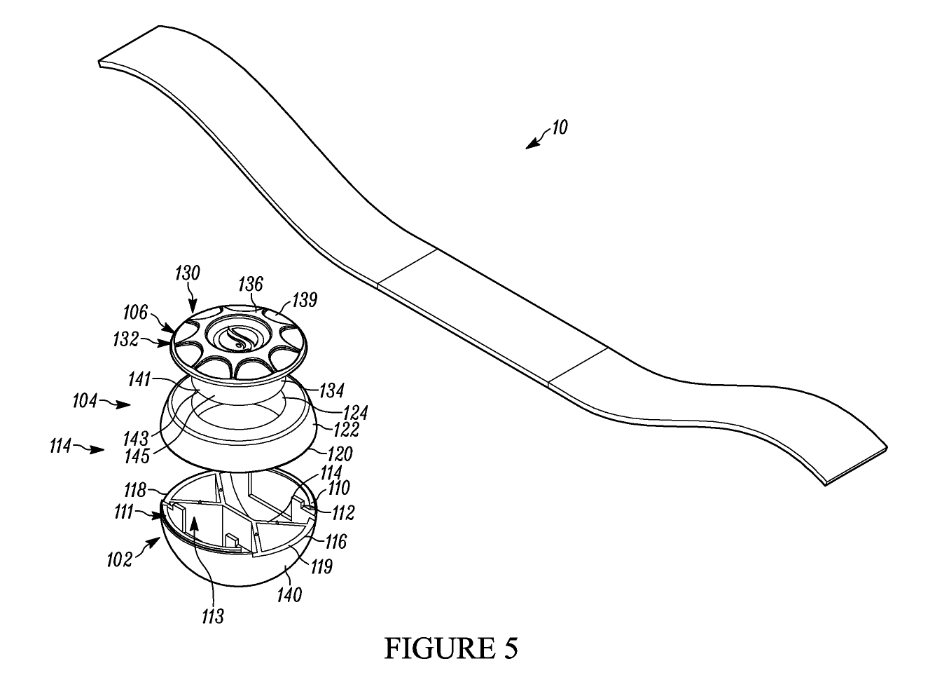

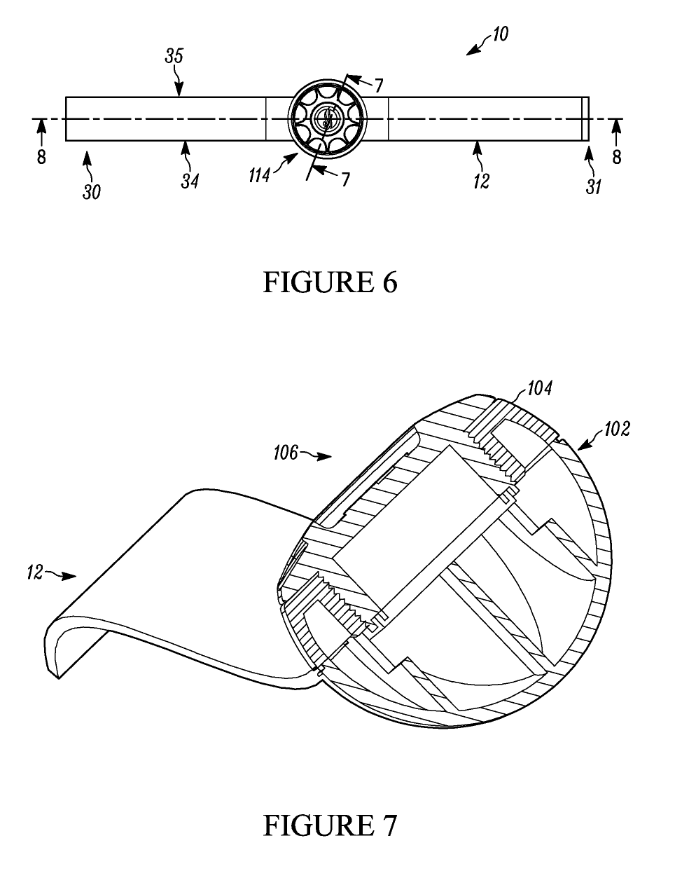

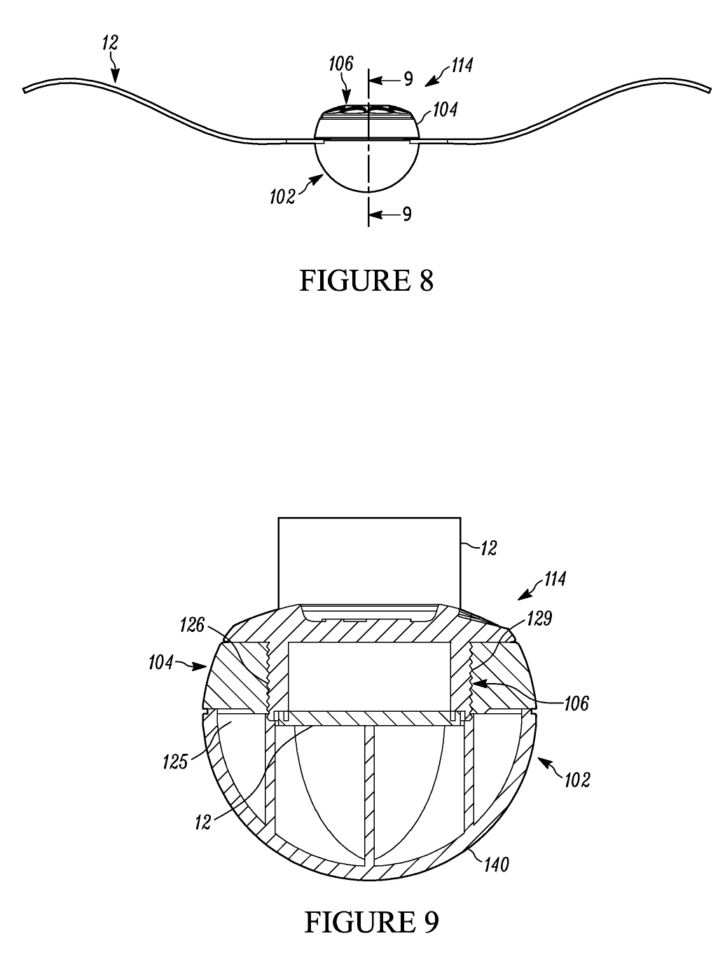

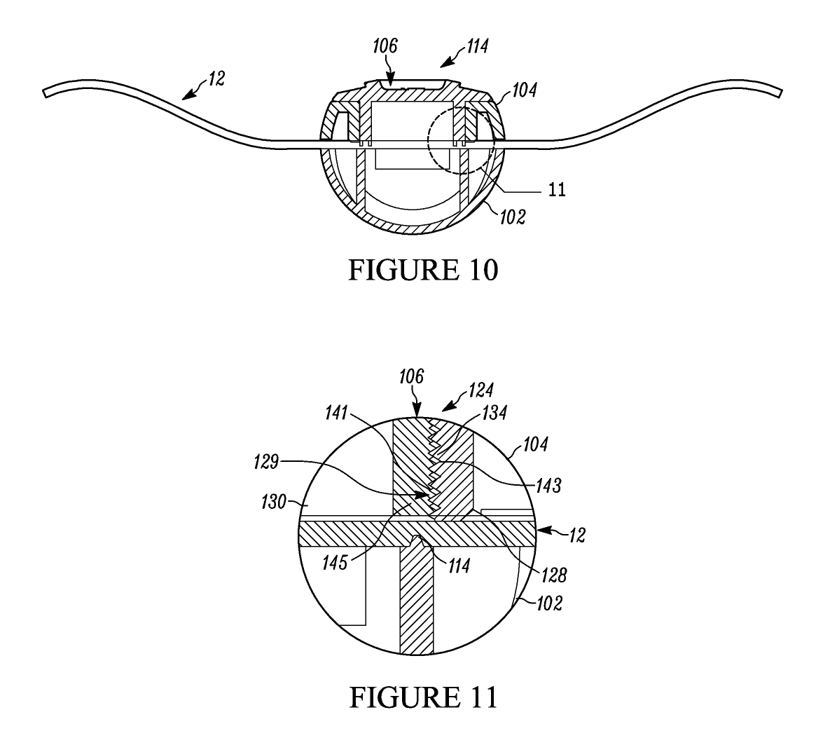

One such configuration of multiple part nodes are disclosed, is shown in FIGS. 5 through 10, collectively, as comprising movement limiting nodes, such as movement limiting node 114. The movement limiting node 114 includes outward node component 102, inward node component 104 and locking component 106. The outward node component 102 includes outward node surface 140, outer rim 110, alignment posts 112 and grasping protrusions 114. The outward node component generally comprises a molded member, preferably a polymeric member, although variations are contemplated.

The outward node surface 140, as described above generally comprises a hemispherical-like configuration. The outer rim 110 generally comprises a circular member that is generally defined at the upper extremity of the hemispherical-like configuration, and generally defines an upper plate or plane 111 (which is of a generally circular configuration). Webbing, such as webbing 113 may extend throughout the inner portion (or cavity) defined by the outward node surface 140 so as to provide the necessary structure and rigidity.

The alignment posts 112 are contained within the bounds of the outer rim, generally, and extend upwardly beyond the outer rim. In the configuration shown, a total of four alignment posts are positioned strategically, to, together with the outer rim, define a passageway 116 that extends across the outward node component, generally parallel to the upper plate 111, and generally centrally therethrough. The passageway includes two opposing openings at each of the first end 118 and the second end 119. The passageway and the openings are configured to receive the harness member therethrough (with the understanding that the inward node component forms a portion of the boundaries of the passageway).

Grasping protrusions 114 are positioned in a spaced apart orientation so as to extend into the passageway 116, and generally positioned so as to align with the lower edge 145 of the depending cylindrical portion 134 of the locking component 106. In the configuration shown, a grasping protrusion is positioned in a plurality of locations on the webbing 113 so as to extend above the upper plate 111 strategically positioned where the webbing corresponds to the lower edge of the depending cylindrical portion of the locking component 106. It will be understood that the grasping protrusion interfaces with the lower edge of the depending cylindrical portion of the locking component to sandwich the harness member therebetween in a joined and retained configuration.

The inward node component 104 is shown as including lower outer rim 120, upper outer rim 122 and inner bore 124. The inward node component generally defines a hoop like member having a generally ring like configuration. The lower outer rim 120 is configured to interface with the outer rim 110 of the outward node component 102. The two can be secured together through an adhesive, a weld, a mechanical fit, or a combination thereof. It is contemplated that such a fit is permanent (that is, generally destructive force is required to separate the same). It is also contemplated that the fit may be releasable, that is the two components may be releasably coupled and detachable as desired. The upper outer rim is generally parallel and offset from the lower outer rim, and both are generally parallel to the upper plate 111 of the outward node component 102, and defines the upper surface of the inward node component. The inward node component has the shape of a segment of a sphere.

The inner bore 124 is generally centrally located and includes an upper end 126 and a lower end 128. The inner bore 124 is generally perpendicular to the upper and lower outer rims. The inner bore 124 includes threads 129 that extend along the inner wall thereof between the first and second ends. It will be understood that the threads may extend entirely between the upper and lower end, or may be positioned only partially therebetween.

The locking component 106 includes body 130 that has upper control member 132 and depending cylindrical portion 134. The upper control member 132 includes upper face 136 and lower surface 138. The upper surface 136 includes a control members 139 which aid a user in grasping and rotating the locking component relative to the inward node component and the outward node component. In the configuration shown, the structures include a plurality of surface variations. The lower surface 138 is configured to abuttingly engage the upper outer rim 122 and upper surface of the inward node component. The upper surface of the upper control member of the locking component and the inward node component together form the inward node surface 142.

The depending cylindrical portion 134 includes threads 141 which extend along the outer surface 143 thereof. The threads 141 are configured to matingly engage with the threads 129 of the inward node component. The depending cylindrical portion, therefore, is sized appropriately so that the threads are configured to engage. The depending cylindrical portion 134 includes lower edge 145 which is configured to contact the harness member positioned within the passageway defined by the outward node component. It will be understood that the locking component 106 can be advanced into the inner bore of the inward node component to essentially tighten and secure the movement limiting node to the harness member, and can be retracted to allow for relative movement of the movement limiting node relative to the harness member. It will be understood that in other configurations, the position of the locking component can be switched so that the locking component interfaces with the outward node component and not the inward node component. In such a configuration, the locking component can be accessed from the outward node component.

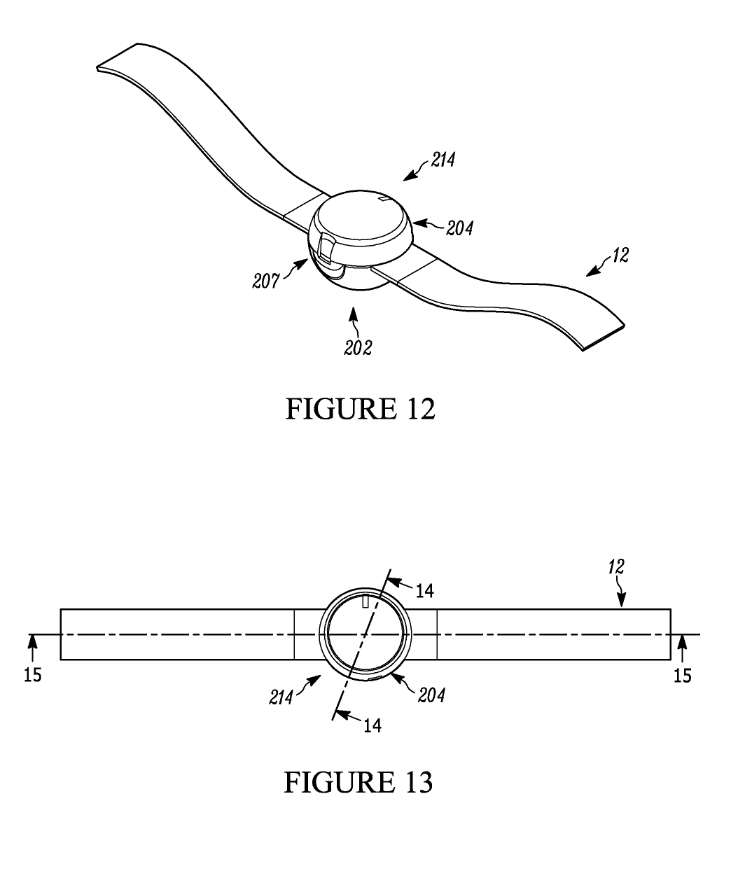

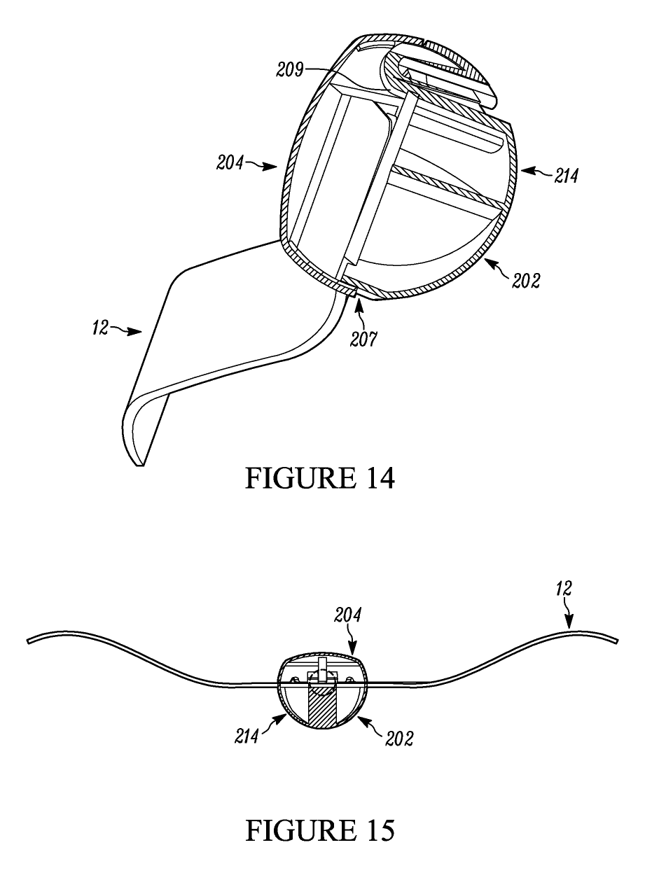

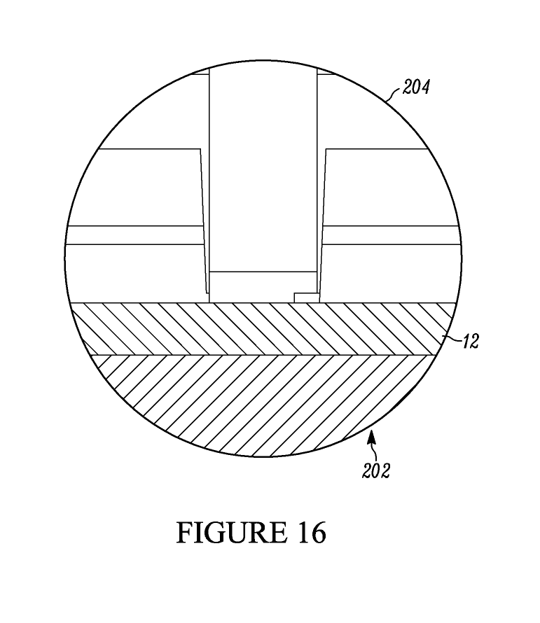

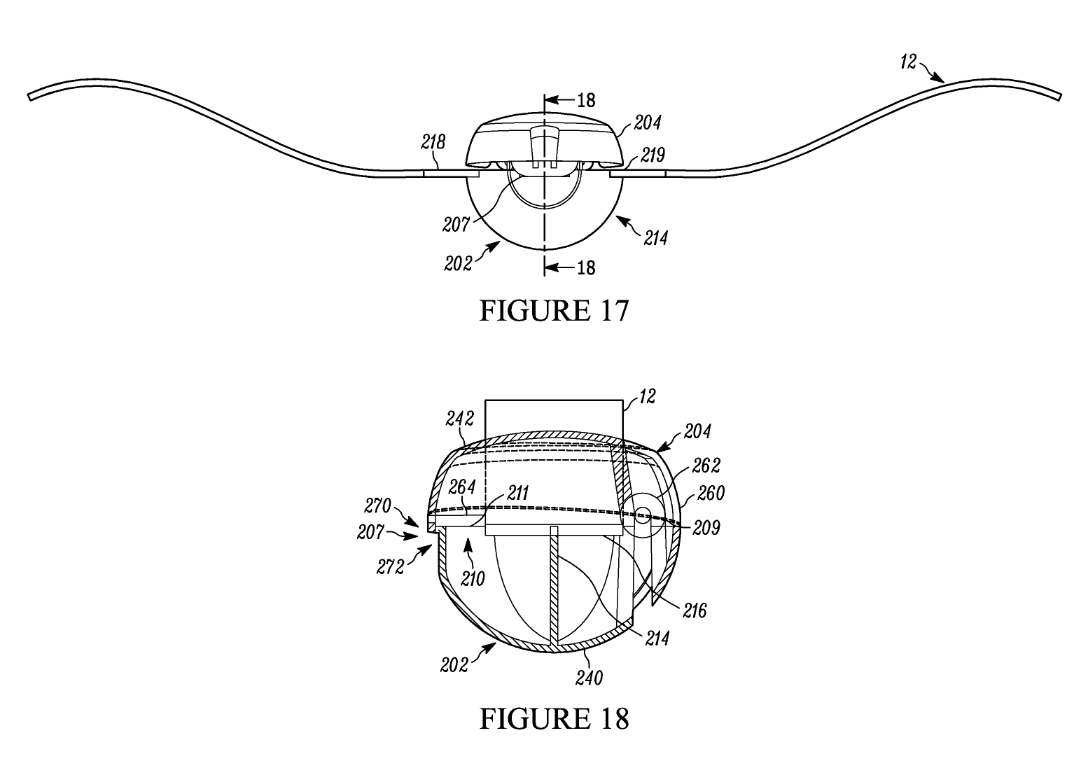

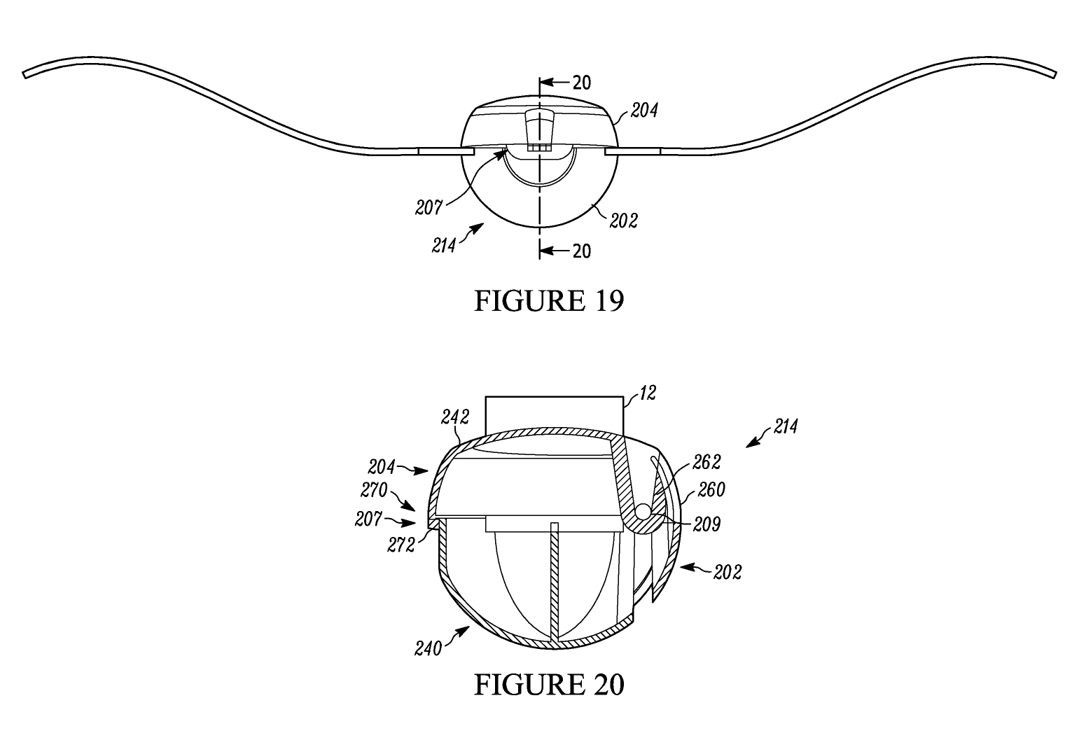

In another configuration shown in FIGS. 11 through 20, movement limiting node 214 is shown as comprising outward node component 202, inward node component 204, latching mechanism 207 and pivoting pin member 209. It will be understood that the harness member 12 is configured to be captured between the outward node component and the inward node component.

The outward node component 202 includes outward node surface 240, outer rim 210, passageway 216, grasping protrusions 214 and transverse pivot opening structure 260. The outward node portion generally comprises a hemispherical configuration. The outer rim 210 is generally substantially planar and defines an upper plate or plane 211. The passageway 216 is positioned into the upper plate or plane and defines first end 218 and second end 219 through which the harness member 12 is positioned.

The grasping protrusions 214 extend upwardly beyond the upper plate or plane 211 and are configured to be directed into the harness member when the movement limiting node is in the capture position (or locked position). In some configurations, in either of the embodiments shown, the grasping protrusions may be eliminated.

The transverse pivot opening portions 260 includes a rib or web portion that extends upwardly beyond the upper plate or plane, and a transverse pivot opening extends therethrough. In the configuration shown, the transverse pivot opening is maintained within the outer rim 210, whereas in other configurations it may be at the outer rim 210 or outside of the outer rim 210.

The inward node component 204 is shown as comprising inward node surface 242, transverse pivot opening portion 262 and lower plane 264. The inward node component is positioned over the upper plate 111 of the outward node component and on the opposite side of the harness member. The transverse pivot opening portion 262 includes a rib member with a transverse opening. It will be understood that the structures are such that in the proper position the transverse opening of the inward node component and the outward node component align, so that the pivoting pin member 209 extends through both of them so as to be pivotably coupled thereto. It will be understood that the pivoting members extend into the inward node component, and it will also be understood that depending on the position of the pivoting pin member, different pivoting ranges and the like are defined. In the configuration shown, the interference of the outward node component and the inward node component outboard of the pivoting pin member contact each other to limit outward pivotal movement of the two components. In the configuration shown, the pivoting pin member 209 is substantially parallel to the position of the harness member within the passageway 216 and on a first side thereof.

The latching mechanism 207 is shown as comprising pivotable latch protrusion 270 on one of the inward and outward nodes (in this case on the inward node component), and capture flange 272 on the other of the inward and outward nodes. The latch mechanism in the configuration shown is directly opposite the pivoting pin member on the other side of the harness member. It will be understood that the pivotable latch protrusion 270 can be directed beyond the capture flange 272 whereupon the pivotable latch protrusion returns to its rest position on the opposite side of the capture flange, thereby locking the two components together. It will be understood that the clamping force on the harness member by the outward node component and the inward node component can be varied by setting the geometry and relationship between the pivotable latch protrusion and the capture flange of the latching mechanism. Additionally, the position and extension of the grasping portion likewise influences the strength and force on the harness member resisting movement.

To release the two components, the user must pivot the pivotable latch protrusion away from the capture flange outwardly, until the latch protrusion extends beyond the capture flange. At such time, the two can be rotated away from each other about the pivot pin member. Once the pivotable latch protrusion extends beyond the flange, it may be released. Generally, the shape of the protrusion and the flange are such that locking can be achieved by pressing the two components toward each other, but that separation generally requires the user to manually pivot the pivotable latch protrusion.

In operation, and with reference to the schematic representations shown in

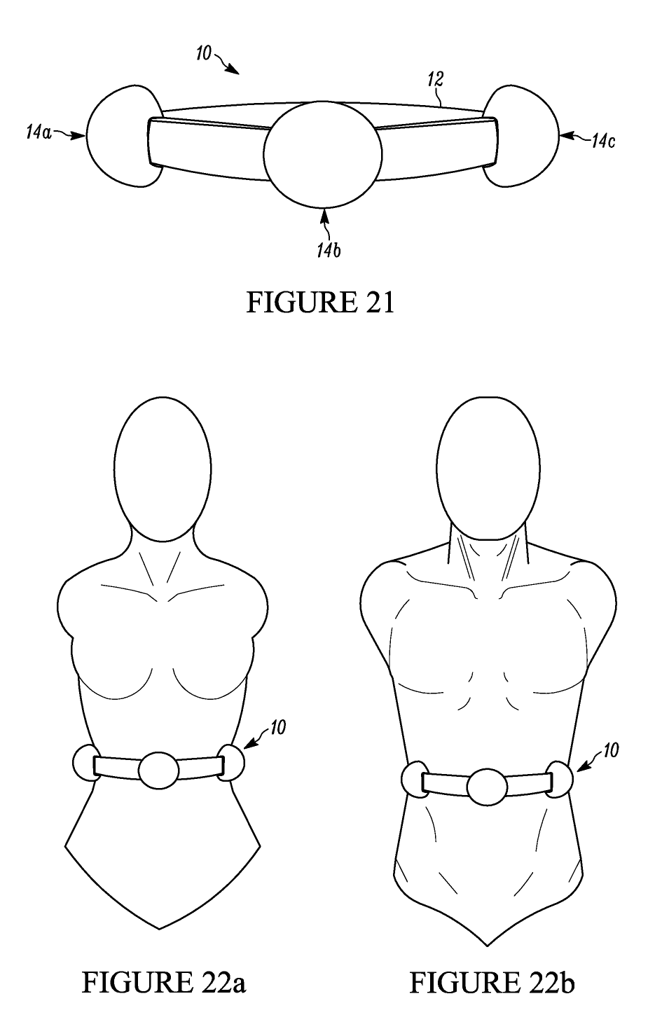

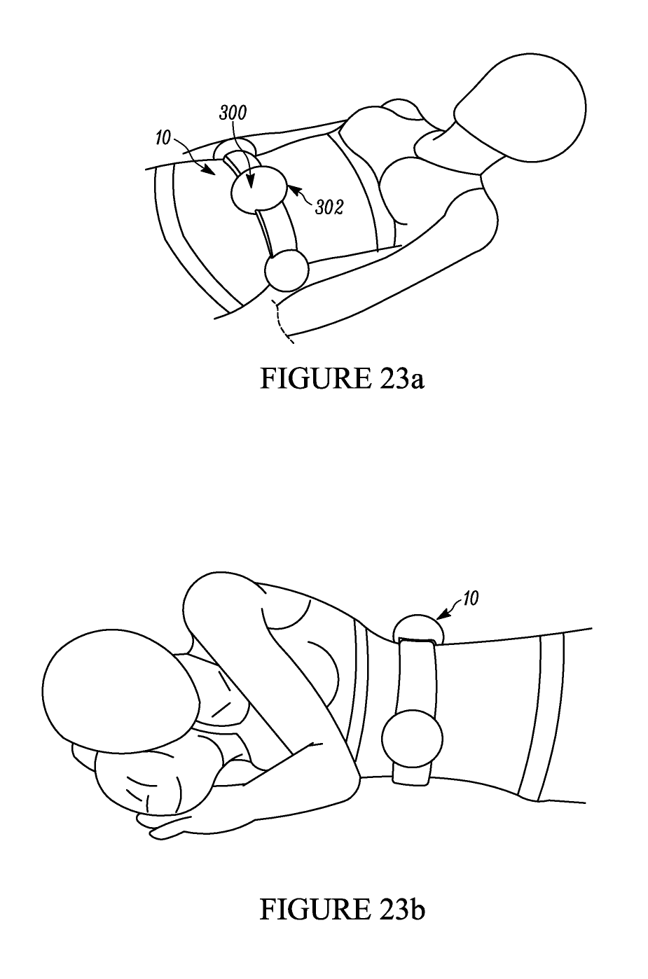

FIGS. 21, 22a, 22b, and 23a-23c, the user first is provided with the harness member, and at least one, and preferably multiple nodes. The user determines if the harness member requires adjustment of the adjustment member so as to be able to be positioned around the user in the general area of the waist of the user. Once any adjustment has been made, the user can address the node position. One such configuration of the sleep aid apparatus is shown in FIGS. 21, and a user wearing the same is shown in FIGS. 22a and 22b.

In particular, the user can position the nodes in such a manner relative to each other so that when in the operating orientation on the user, the nodes define the boundaries of the desired user position when sleeping. That is, the nodes are separated from each other as desired, and the manner in which they are affixed is disclosed above with respect to the different configurations. The apparatus is then installed on the user so that the nodes fall on either side of the surface that the user wishes to rest upon. An additional node can be positioned near the center of a surface upon which sleeping is not desired.

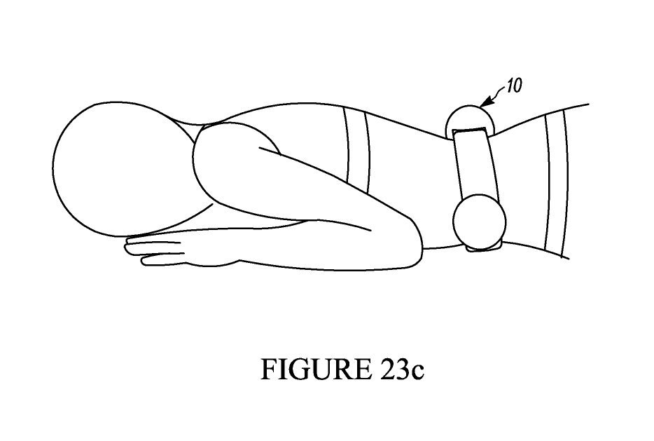

When the user lays down, in the desired position, the nodes will be positioned on either side with an additional node positioned opposite the surface. It will be understood that the nodes are difficult to roll over, and, as such, the nodes preclude the user from turning onto another side (ostensibly, any side other than the desired side). In the event that the user overcomes the node and rolls over the node, the user may encounter the remaining node, which will be uncomfortable and will generally direct the user back into the desired orientation. With reference to FIGS. 23a through 23c, the nodes can be positioned in different orientations depending on the surface upon which the user desires to sleep. For example, in FIG. 23a, the nodes are positioned so that the user sleeps on her back. Whereas, in FIG. 23b, the nodes are positioned so that the user sleeps on her side. Finally, in FIG. 23c, the nodes are positioned so that the user sleeps on her stomach/front. These configurations are for illustrative purposes, and are not deemed limiting.

From time to time, different users may desire to use the device, or the user may wish to adjust the position of the nodes so as to better suit the needs of the user toward achieving the desired sleeping position. Should adjustment be desired, the user can pull the movement limiting node relative to the harness member to adjust the relative position of the node and the harness member. It will be understood that each of the different nodes can be adjusted independently of each other, and in the manners disclosed above, depending on the particular configuration. It will also be understood that the movement limiting nodes may include a vibration element (such a vibration element 300 in FIG. 23a) therein, which is configured to vibrate if the user attempts to overcome the node and roll over the node. This prompts the user to return to the prior configuration. It is further contemplated that the node may include a communication device 302 coupled to the vibration element, which is configured to wirelessly communicate with an outside computing device (such as a smart phone or the like through a communication standard such as Bluetooth), so that data pertaining to triggered vibrations and the like can be transmitted to the device for further analysis at a later date.

The foregoing description merely explains and illustrates the invention and the invention is not limited thereto except insofar as the appended claims are so limited, as those skilled in the art who have the disclosure before them will be able to make modifications without departing from the scope of the invention.

What is claimed is:

1. A sleep aid apparatus comprising:
a harness member comprising a body with a first end and a second end and defining a length and a width, and, a closure member configured to couple the first end and the second end in releasable engagement; and
at least one movement limiting node positionable along the harness member, the at least one movement limiting node including an outward node surface extending from an outer surface of the body of harness member.

2. The sleep aid apparatus of claim 1 wherein the at least one movement limiting node includes an outward node surface extending from an outer surface of the body of the harness member, and inward node surface extending from an inner surface of the body of the harness member.

3. The sleep aid apparatus of claim 2 wherein a passageway is defined between the outward node surface and the inward node surface, configured to receive the harness member therethrough.

4. The sleep aid apparatus of claim 3 wherein the at least one movement limiting node releasably lockingly engages the harness member, to, in turn, preclude the relative movement of the at least one movement limiting node and the harness member.

5. The sleep aid apparatus of claim 1 wherein the at least one movement limiting node comprises three movement limiting nodes, positionable in a space apart orientation.

6. The sleep aid apparatus of claim 1 wherein the outward node surface comprises a generally hemispherical configuration.

7. The sleep aid apparatus of claim 1 wherein the closure member includes a first component positionable proximate the first end of the body of the harness member, and a second component positionable proximate the second end of the body of the harness member, wherein the first component and the second component are releasably attachable to each other.

8. The sleep aid apparatus of claim 1 wherein the at least one movement limiting node further comprises:
an outward node component having the outward node surface;
an inward node component pivotably coupled to the outward node component, the outward node component and the inward node component defining a passageway therebetween for the harness member, wherein pivoting of the outward node component relative to the inward node component provides selective access to the passageway therebetween, and directs the outward node component and the inward node component between an open orientation and a harness member retaining orientation; and
a latching mechanism configured to releasably maintain the outward node component and the inward node component in a harness member retaining orientation.

9. The sleep aid apparatus of claim 8 wherein the outward node component and the inward node component are pivotable about an axis that is substantially parallel to the passageway for the harness member.

10. The sleep aid apparatus of claim 9 wherein the axis is substantially maintained within the footprint of the inward node component and the outward node component.

11. The sleep aid apparatus of claim 10 wherein interference between the inward node component and the outward node component on a side opposite the axis from the passageway precludes further pivoting thereof away from the harness member retaining orientation.

12. The sleep aid apparatus of claim 8 wherein the latching mechanism includes a pivotable latch protrusion on one of the inner node component and the outer node component, and a capture flange on the other of the inner node component and the outer node component.

13. The sleep aid apparatus of claim 8 wherein the outward node component further includes at least one grasping protrusion that extends into the passageway which is configured to extend into the harness member when in the harness member retaining orientation.

14. The sleep aid apparatus of claim 1 wherein the at least one movement limiting node further comprises:
an outward node component including the outward node surface, and an outer rim;
an inward node component including a lower outer rim, and an upper outer rim, and including an inner bore extending therethrough, the inner bore being threaded along at least a portion thereof, the outward node being coupled to the inward node, with the inward node and the outward node cooperatively forming a passageway for the harness member;
a locking component having a body with an upper control member and a depending cylindrical portion, the depending cylindrical portion including threads on the outer surface thereof, and a lower edge opposite the upper control member, wherein the depending cylindrical portion is threadedly coupled to the threads of the inner bore of the inward node component, wherein continued threading will direct the lower edge into contact with the harness member, sandwiching the harness member between the lower edge and the outward node component.

15. The sleep aid apparatus of claim 14 wherein the outward node component includes a grasping protrusion, the grasping protrusion positioned on the outward node component so as to correspond to the lower edge of the depending cylindrical portion, to, in turn, provide a point of contact with the lower edge.

16. The sleep aid apparatus of claim 1 further including a vibration element positioned within the movement limiting node, the vibration element activated by force upon the outward node component.

17. The sleep aid apparatus of claim 16 further including a communication element coupled to the vibration element, the communication element configured to communicate any activation of the vibration element through a wireless communication.
